(19) United States
(12) Patent Application Publication
(10) Pub. No.: US 2015/0164781 A1
(43) Pub. Date: Jun. 18, 2015

(54) Personal Care Products Containing Extracts of Chinese Lantern (Physalis alkekengi)

(71) Applicant: Kemin Industries, Inc., Des Moines, IA (US)

(72) Inventors: Shuting Wei, Ames, IA (US); Megan Winters, Urbandale, IA (US); Satish Nayak, West Des Moines, IA (US)

(21) Appl. No.: 14/568,707

(22) Filed: Dec. 12, 2014

Related U.S. Application Data
(60) Provisional application No. 61/915,300, filed on Dec. 12, 2013.

(51) Int. Cl. A61K 8/97; A61Q 19/00

(52) U.S. Cl. CPC A61K 8/97; A61Q 19/00; A61K 2800/10

(57) ABSTRACT

A method of reducing skin damage and/or providing health or cosmetic benefits in a subject, comprising application of a composition containing an effective amount of extracts of Chinese lantern (Physalis alkekengi) to the skin of the subject.

This application claims priority to U.S. Patent Application Ser. No. 61/915,300, filed Dec. 12, 2013, which is incorporated herein by this reference.

### BACKGROUND OF THE INVENTION

The present invention relates generally to personal care products containing extracts of Chinese lantern (Physalis alkekengi) and, more specifically, to the addition of extracts of Chinese lantern to personal care products to protect the skin against damage due to exposure to ultraviolet radiation, provide skin soothing effects, improves skin conditioning and reduce the effects of skin aging.

Skin experiences constant exposure to environmental insult, including ultraviolet radiation (UV) exposure. The results of this exposure can be the accumulation of senescent keratinocytes. These keratinocytes can have a negative impact on the structure and function of the skin. Specifically, UV radiation on the skin can result in the formation of intracellular reactive oxygen species (ROS), which can cause DNA damage. This DNA damage acts as the stimulus to induce cellular senescence. The visual result of this process is aging. The first defense against this response is to utilize the antioxidants that are found in our bodies provided by healthy diet. Another opportunity to reduce the impact of this exposure is to utilize antioxidants applied topically on the skin via personal care products. Antioxidants have the ability to quench free radicals caused by the environment before they can cause a downstream effect.

Skin can undergo different types of environmental and physical stress on a daily basis, including exposure to irritating chemicals or UV radiation. As the body's natural response mechanism, epidermal keratinocytes can release a vast array of cytokines, such as interleukin 6, 8, 1alpha (IL-6, IL-8 and IL-1α) and Tumor necrosis factor alpha (TNF-α). These cytokines can be used as the body's immune response to environmental exposure and are associated with the development of the irritation symptoms. These irritation symptoms can cause skin to become red and sore. Antioxidants help aid in soothing skin irritation.

### SUMMARY OF THE INVENTION

Extracts of Chinese lantern have been discovered to have antioxidant effects that impact skin care issues. Included benefits are a decrease in inflammatory markers following exposure of the skin to UV radiation. The addition of these extracts of Chinese lantern to personal care products provides a methodology for providing skin care benefits to users of the products, including anti-aging effects.

A purpose of the present invention is to provide personal care products containing extracts of Chinese lantern to provide beneficial effects to users of the products.

Another purpose of the present invention is to provide personal care products containing extracts of Chinese lantern for use by persons at risk for skin damage.

Yet another purpose of the present invention is to provide a method of reducing skin damage in a subject, comprising application of a composition containing an effective amount of extracts of Chinese lantern to the skin of the subject.

Still a further purpose of the present invention is to provide a method of reducing the levels of an inflammatory marker in a subject, comprising application of a composition containing an effective amount of extracts of rosemary to the skin of the subject.

Yet a further purpose of the present invention is to provide a method of reducing the levels of one or both of the inflammatory markers IL-6, IL-8, IL-1α and TNFα.

These and other purposes of the present invention will be understood by those skilled in the art upon a review of this specification, the associated figures and the appended claims.

### BRIEF DESCRIPTION OF THE DRAWINGS

FIG. 1 is a chart of the results of the MTT assay of Example 1.

FIG. 2 is a chart of the results of the IL-6 assay of Example 1.

FIG. 3 is a chart of the results of the IL-8 assay of Example 1.

FIG. 4 is a chart of the results of the IL-1α assay of Example 1.

FIG. 5 is a chart of the results of the TNFα assay of Example 1.

FIG. 6 is a chart of the inhibition of 0.05 mg/mL of DPPH by trolox (IC50=0.0010%).

FIG. 7 is a chart of the inhibition of 0.05 mg/mL of DPPH by LumiSalis SE.

### DETAILED DESCRIPTION OF PREFERRED EMBODIMENTS

Chinese lantern (Physalis alkekengi) is a herbaceous perennial plant native to Asia that produces an orange to red fruit enclosed in a larger, bright orange to red papery basal calyx. Chinese lantern is a popular ornamental plant and has been used in traditional medicine as a diuretic, antiseptic, liver corrective and sedative. Chinese lantern calixes have a high level of antioxidant compounds, including carotenoids.

As used herein, “reducing” means treating, ameliorating, reducing the adverse appearance of, reducing the severity of, or reducing the adverse effects of.

As used herein, “inflammatory markers” means substances produced by the body that may be indicative of or precede inflammation, and includes but is not limited to interleukin-6 (IL-6), interleukin-8 (IL-8), interleukin 1-alpha (IL-1α) and tumor necrosis factor alpha (TNFα).

As used herein, “skin damage” includes but is not limited to fine and coarse wrinkles, irregular pigmentation, large freckle-like spots called lentigines, a yellowish complexion, and a leathery, rough skin texture. Skin damage also includes: (a) dry skin, wherein sun-exposed skin can gradually lose moisture and essential oils, making it appear dry, flaky and/or prematurely wrinkled, even in younger people; (b) sunburn, which is the common name for the skin damage or injury that appears immediately after the skin is exposed to UV radiation, mild sunburn causes only painful reddening of the skin, but more severe cases can produce tiny fluid-filled bumps (vesicles) or larger blisters, and (c) actinic keratosis which is a tiny bump that feels like sandpaper or a small, scaly patch of sun-damaged skin that has a pink, red, yellow or brownish tint; unlike suntan markings or sunburns, an actinic keratosis does not usually go away unless it is frozen, chemically treated or removed by a doctor; an actinic keratosis develops in areas of skin that have undergone repeated or long-term exposure to the sun's UV light, and it is a warning sign of increased risk of skin cancer.

As used herein, “therapeutically effective amount” means the amount of a compound or composition or derivatives thereof of the present invention is an amount that, when administered to a subject, will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the condition, and the therapeutics or combination of therapeutics selected for administration, and the mode of administration. The skilled worker can readily determine the effective amount for a given situation by routine experimentation. In one embodiment, the extract of Chinese lantern as described herein is added to a personal care product for application to the skin in a therapeutically effective amount when used as directed.

As used herein, “treatment or treating” means intervention in an attempt to alter the natural course of the individual, animal or cell being treated, and may be performed either for prophylaxis or during the course of clinical pathology. Desirable effects include preventing occurrence or recurrence of disease, alleviation of symptoms, diminishment of any direct or indirect pathological consequences of the disease, lowering the rate of disease progression, amelioration or palliation of the disease state, and remission or improved prognosis. A condition or subject refers to taking steps to obtain beneficial or desired results, including clinical results. Beneficial or desired clinical results include, but are not limited to, reduction, alleviation or amelioration of one or more symptoms associated with skin damage.

In preferred embodiments of the present invention, the dosage of an extract of Chinese lantern ranges from 0.001% by weight of a personal care product to 10% by weight of a personal care product and all values between such limits, including, for example, without limitation or exception, 0.002%, 0.003%, 0.004%, 0.01%, 0.03%, 0.06%, 0.09%, 0.1%, 0.25%, 0.7%, 1%, 2%, 3%, 4%, 4.15%, 6.63%, and 9.87%. Stated another way, in preferred embodiments of the invention, the dosage can take any value “ab.cde wt %” wherein a is selected from the numerals 0 and 1, and b, c, d and e are each individually selected from the numerals 0, 1, 2, 3, 4, 5, 6, 7, 8 and 9 with the exception that a, b, c, d and e cannot all be 0.

### EXAMPLE 1

### Fibroblast IL-6, IL-8, IL-1α and TNFα

### Purpose

This assay procedure can be used to screen materials for their ability to reduce ultraviolet light (280-315 nm, usually referred to as UVB)—induced increases in IL-6, IL-8, IL-1α and TNFα using cultured fibroblasts as the model.

### Preparation of Test Material

Fresh Chinese lantern berries were freeze-dried. The obtained dried berries were then ground to produce a powder. This powder was used for extraction of oil. Extraction was conducted using supercritical carbon dioxide. The extraction was conducted using the following conditions: Temperature, 45° C.; pressure, 300 bar; CO₂ flow rate, 100 g/min; and solvent to feed ratio, 70. The obtained extract was an orange-colored oil. This oil (CLSC) was used in the following bioassays at concentrations of 0.01%, 0.005%, and 0.001%.

### Summary of Test Method

Exposure of skin cells to UVB can result in an inflammatory response associated with the release of inflammatory markers such as IL-6, IL-8, IL-1α and TNFα. The release of inflammatory mediators can have detrimental effects on the appearance and function of the skin. Therefore, material which can prevent these UVB induced responses can be beneficial as cosmetic ingredients.

In this study human dermal fibroblasts were grown in culture and treated for 24 hours after the UVB exposure. At the end of the incubation period the cell culture media was collected and assayed for IL-6, IL-8, IL-1α and TNFα using ELISA based methods. Changed in cell viability was also assessed using an MTT assay.

### Materials and Methods

### Fibroblast Cell Culture

Human dermal fibroblasts were seeded into 24-well plates in Fibroblast Growth Medium (FGM) and grown at 37±2° C. and 5±1% CO₂ until confluent with a media change every 48 to 72 hours as needed. Once the cells were confluent they were treated with the test materials after the UVB exposure. During the 24 hours prior to the UVB exposure the cells were treated with DMEM media alone. For the UVB exposure the cell culture media was replaced with PBS and the cells were irradiated with 40 mJ/cm² UVB. After the UVB exposure the cells were treated with the test materials prepared in DMEM. At the end of the incubation period the cell culture media was collected and assayed for IL-6, IL-8, IL-1α and TNFα while changes in cell viability was determined using an MTT assay.

### MTT Assay

After the UVB incubation, the cell culture medium was removed (see above) and the fibroblasts were washed twice with PBS to remove any remaining test material. After the final wash, 500 μl of DMEM supplemented with 0.5 mg/ml MTT was added to each well and the cells were incubated for 1 hour at 37±2° C. and 5±1% CO₂. After the incubation, the DMEM/MTT solution was removed and the cells were washed again once with PBS and then 0.5 ml of isopropyl alcohol was added to the well to extract the purple formazin crystals. Two hundred microliters of the isopropyl extracts was transferred to a 96-well plate and the plate was read at 540 nm using isopropyl alcohol as a blank.

### Preparation of ELISA Plates (IL-6, IL-8, IL-1α and TNFα)

The ELISA plates were prepared by diluting the appropriate capture antibody in PBS. Next, 100 μl of the diluted capture antibody was added to the wells of a 96-well ELISA plate and the plate was incubated overnight at room temperature. On the following day the plate was washed three times with 300 μl wash buffer (0.05% Tween 20 in PBS) and then blocked by adding 300 μl of blocking buffer (1% BSA in PBS) to each well. The plate was incubated with the blocking buffer for at least one hour. After the incubation the blocking buffer was removed and the plate was washed three times as described above.

### ELISA Procedure

A series of standards was prepared and 100 μl of each of these standards was dispensed into two wells (duplicates) in the appropriate 96-well plate. Subsequently, 100 μl of each sample was added to additional wells and the plate was incubated for two hours at room temperature. After the incubation the plate was washed three times as described above. Once the last wash was removed, 100 μl of a biotin conjugated detection antibody was added. After incubating the plate for two hours at room temperature the plate was washed again as described above. 100 μl of HRP-streptavidin was then added to each well and the plate was incubated for 20 minutes at room temperature. Once the last wash was removed, 100 μl of substrate solution (hydrogen peroxide +tetramethylbenzidine as a chromagen) was added to each well. Once a sufficient level of color development had occurred, 50 μl of stop solution (2N sulfuric acid) was added to each well and the plate was read at 460 nm.

### Results

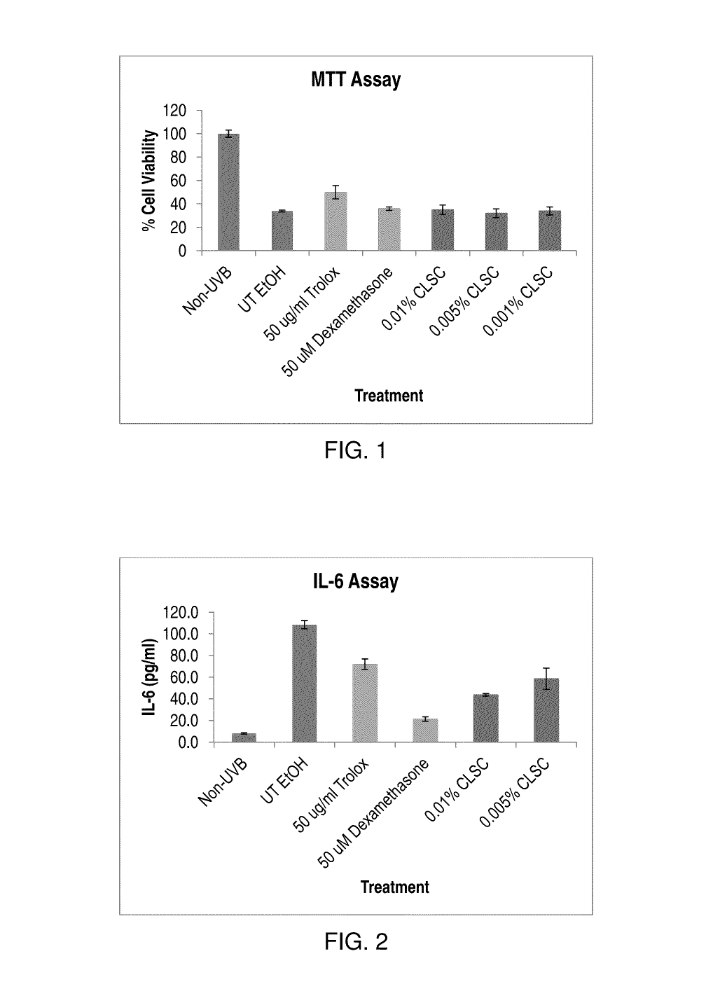

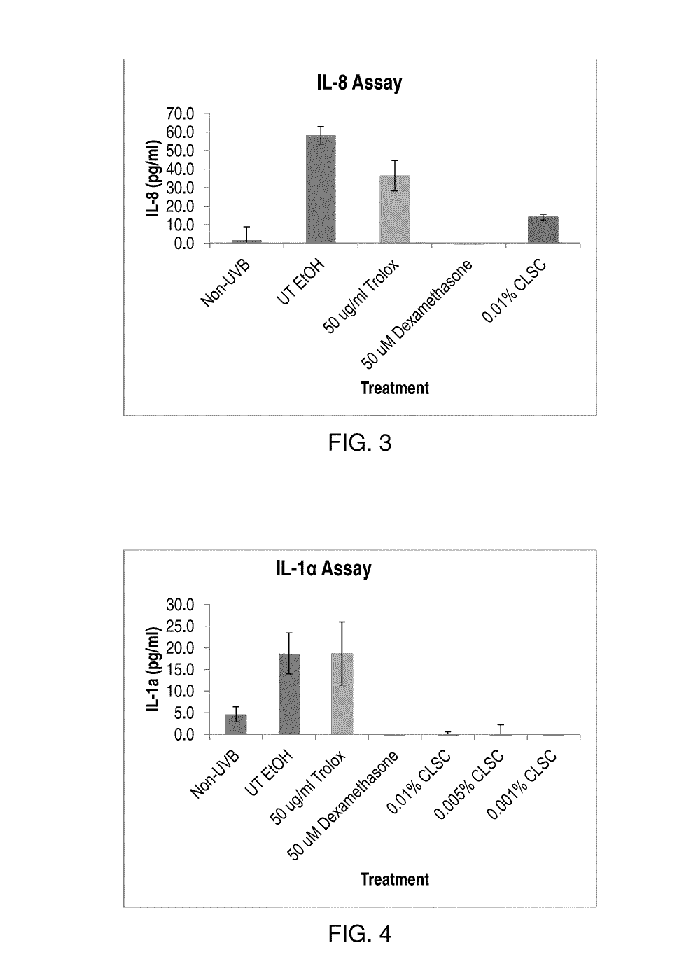

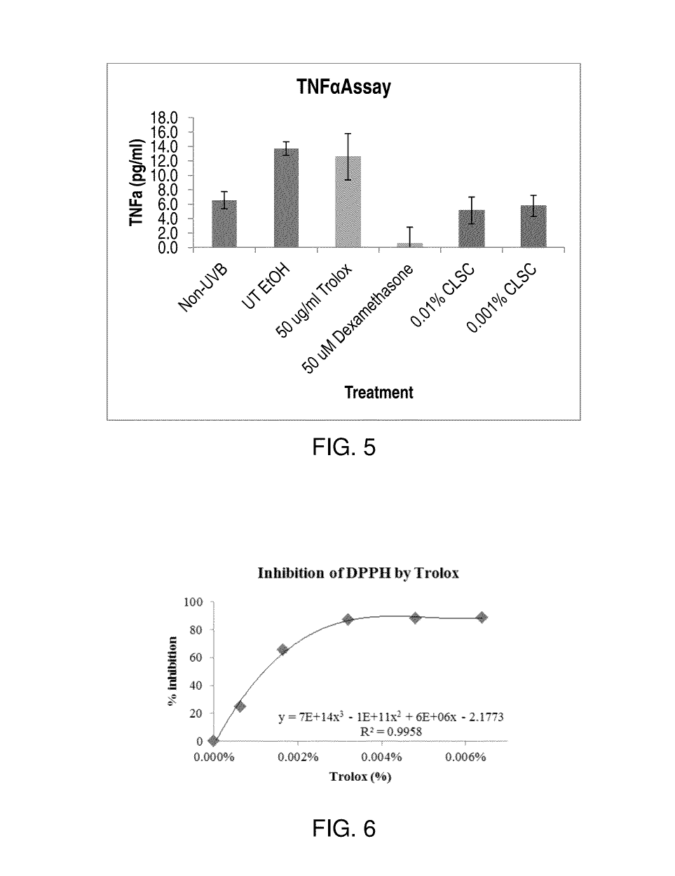

The results for the MTT assay are presented in FIG. 1. The values for this assay are expressed as mean viability ±the standard deviation. The results for the IL-6, IL-8, IL-1α and TNFα assays are presented in FIGS. 2-5, respectively. The values for these assays are presented as mean concentration±standard deviation.

### Discussion

UVB irradiation of the fibroblasts resulted in a significant decrease in the number of viable cells, along with a significant increase in all of the proteolytic and inflammatory markers measured in this study. The treatments with the test materials were not associated with any further decrease in the number of viable cells.

When CLSC was added to the fibroblasts after the UVB exposure there was no a further decline in the number of viable cells and this material was observed to significantly decrease the release of IL-6 and IL-8, IL-1α and TNFα.

### EXAMPLE 2

### Characterization of the Antioxidant Activity of Chinese Lantern Extract by a Microtiter Plate DPPH Assay

### Materials and Methods

### Reagents.

HPLC grade reagent alcohol was obtained from Fisher Scientific, catalog number A995-4. 2,2-Diphenyl-1-picrylhydrazyl (DPPH) was from Fisher Scientific, catalog number AA4415003. Trolox was from Fisher Scientific, catalog number 05-402-25. Test material was prepared as in Example 1 (LumiSalis™ SE lot 592142P5, 592142P8 and 592142P9).

### Equipment.

Absorbance was measured using a Molecular Devices SpectraMax M5e plate reader (KH51-001) with a clear, flat-bottom 96-well plate, Advangene Life Science Plasticware catalog number CC plate-PS-965-F-C-S. Masses were measured with a Mettler Toledo analytical balance (Model X5204). A multichannel automatic pipette (100-μL; Fisher Scientific catalog number FBE800300), single channel pipettes (200-μL, 1000-μL and 5000-μL; Thermo Scientific catalog number 4642080, 4642090 and FBE 0500 respectively) were utilized in this assay.

### Assay Procedure.

The procedure was as follows. Briefly, an ethanolic DPPH working solution (0.075 mg/mL) was prepared. Six Trolox solutions (used as a control) ranging from 0 to 0.006% were prepared. Sample solutions were prepared in triplicate by dissolving LumiSalis SE in ethanol and diluting to six different concentration ranging from 0 to 0.35%. For sample background analysis, 75 μL of sample solution were pipetted into a clear, flat-bottom 96-well plate. A zero standard of reagent alcohol (75 μL) was also plated. 150 μL of reagent alcohol was added to all blank and sample wells. For sample analysis, 75 μL of sample solution were pipetted into a clear, flat-bottom 96-well plate. A zero standard of reagent alcohol (75 μL) was also plated. DPPH solution (150 μL) was added to all blank and sample solutions. The controls and samples were all plated in triplicate and the average absorbance value was used for results calculations. The plate was shaken and incubated at 25° C. in the microplate reader for five minutes. The absorbance was measured at 517 nm.

Calculations.

The sample concentrations (x-axis) are plotted against their respective relative average absorbance value (subtracted the average absorbance of the background from the average absorbance) at 517 nm (y-axis) and the best fit line (a polynomial fit) is calculated. The 1050 (50% inhibition) of each control and sample were calculated based on the polynomial regression line equation.

### Results

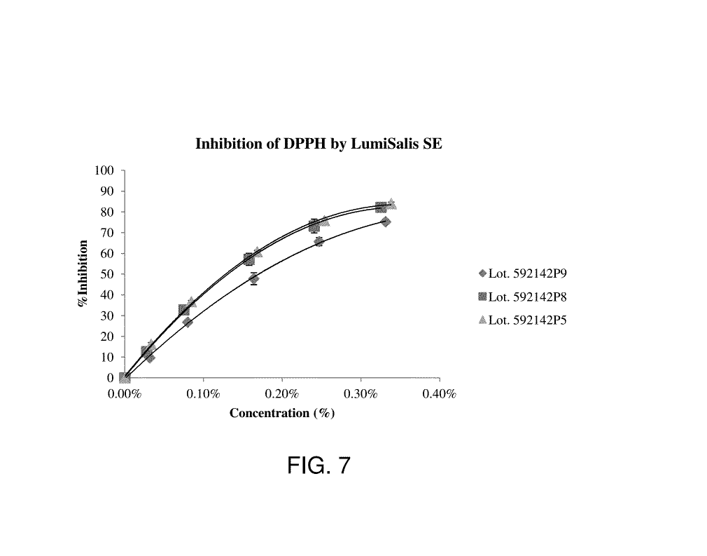

The antioxidant activity of trolox by DPPH assay is shown in FIG. 6. The calculated 1050 of trolox for DPPH is 0.0010%. Three lots of LumiSalis SE were tested by DPPH assay as shown in FIG. 7. The regression equations and 1050 of these three lots are shown in Table 1. These results indicate that LumiSalis SE has antioxidant activity with 1050 of 0.14±0.02% on the DPPH assay.

TABLE 1
Calculation of IC50 of LumiSalis SE on DPPH assay

| | | | | | 95% |
|---|---|---|---|---|---|
| | | | | Aver- | Confi- |
| LumiSalis | | | | age | dence |
| SE | Regression Equation | R² | IC50 | IC50 | interval |
| 592142P9 | y = −4E + 6X² + | 0.9993 | 0.17% | 0.14% | 0.02% |
| | 36895x − 0.564 | | | | |
| 592142P8 | y = −7E + 6X² + | 0.9995 | 0.13% | | |
| | 46475x + 0.4755 | | | | |
| 592142P5 | y = −7E + 6X² + | 0.9993 | 0.13% | | |
| | 46920x + 0.8947 | | | | |

### Discussion

The results of the DPPH assay show that LumiSalis (CLSC) is a potent antioxidant which, when added to personal care products and applied to the skin of a user can be expected to provide the usual benefits seen with antioxidants applied to the skin, such as helping protect the skin against sun damage and skin cancer and reverse some of the discoloration and wrinkles associated with aging. LumiSalis can be expected to speed up the skin's natural repair systems and inhibit further damage.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method of reducing skin damage in a subject, comprising application of a composition containing an effective amount of extracts of Chinese lantern (Physalis alkekengi) to the skin of the subject.

2. A method of reducing the levels of an inflammatory marker in a subject, comprising application of a composition containing an effective amount of extracts of Chinese lantern (Physalis alkekengi) to the skin of the subject.

3. The method of claim 2, wherein the inflammatory marker is selected from the group consisting of IL-6, IL-8, IL-1α and TNFα.

4. A method of treating skin damage in a subject, comprising application of a composition containing an effective amount of extracts of Chinese lantern (Physalis alkekengi) to the skin of the subject.
